(19) United States
(12) Patent Application Publication
(10) Pub. No.: US 2023/0190787 A1
(43) Pub. Date: Jun. 22, 2023

(54) SYSTEM AND METHOD FOR TREATING CARDIAC TOXICITY VIA GUT MICROBIOTA

(71) Applicants: Firoz Anwar, Jeddah (SA); Turky Omar Asar, Jeddah (SA); Mohammed A Almujtaba, Jeddah (SA); Muhammad Shahid Nadeem, Jeddah (SA); Ryan Adnan Sheikh, Jeddah (SA); Abdulbasit I. Al-Sieni, Jeddah (SA); Maged Al Ansari, Jeddah (SA); Salma Naqvi, Ajman (AE); Fahad A. Al-Abbasi, Jeddah (SA); Vassem Ahmad Ansari, Lucknow (IN); Hisham N. Altayb, Jeddah (SA); Sultan Alhayyani, Jeddah (SA); Vikas Kumar, Allahabad (IN); Mateen Hasan Khan, Pakbara (IN)

(72) Inventors: Firoz Anwar, Jeddah (SA); Turky Omar Asar, Jeddah (SA); Mohammed A Almujtaba, Jeddah (SA); Muhammad Shahid Nadeem, Jeddah (SA); Ryan Adnan Sheikh, Jeddah (SA); Abdulbasit I. Al-Sieni, Jeddah (SA); Maged Al Ansari, Jeddah (SA); Salma Naqvi, Ajman (AE); Fahad A. Al-Abbasi, Jeddah (SA); Vassem Ahmad Ansari, Lucknow (IN); Hisham N. Altayb, Jeddah (SA); Sultan Alhayyani, Jeddah (SA); Vikas Kumar, Allahabad (IN); Mateen Hasan Khan, Pakbara (IN)

(21) Appl. No.: 18/058,828

(22) Filed: Nov. 25, 2022

(51) Int. Cl. A61K 33/00; A61K 49/00; C12Q 1/689; G01N 1/31

(52) U.S. Cl. CPC A61K 33/00; A61K 49/0008; C12Q 1/689; G01N 1/312

(57) ABSTRACT

The system comprises a collection unit for procuring doxorubicin, diethyl ether, alkaline water and a plurality of subject animals; a pre-processor unit for acclimatizing collected plurality of subject animals for 7 days standard laboratory condition; a classification processor unit for dividing subject animals into four groups; a treating unit for feeding subject animals with standard semi purified diet along with normal drinking water and standard semi purified diet along with Zam water p.o at libitum, or injecting with Dox i.p. on first day of protocol fed with standard semi purified diet along with normal drinking water or Dox i.p. on first day of protocol thereby treating with Zam water p.o at libitum for 28 days; a monitoring processor unit for monitoring physiological and behavioral changes of plurality of subject animals; and an analysis processor unit for estimating a set of biochemical parameters and stool sample of the plurality of subject animals.

### FIELD OF THE INVENTION

The present disclosure relates to therapeutic potential and benefits of Alkaline water (Zam), in more details, a system and method for treating cardiac toxicity via gut microbiota.

### BACKGROUND

From the time immortal, water is considered to be a natural healer for several ailments. Alkaline water contains a wide range of therapeutic and pharmacological properties like cardio-protective, anti-obesity, anti-atherosclerosis etc. Consumption of drinking water from different origins is subjected to various chemical and physical treatments, before reaching the final consumer. Common treatments include flocculation, sedimentation, filtration and disinfection. The combination of the environmental conditions at source of origin and at various steps of processing including distribution, storage and transport often renders the drinking water with multiple and distinct physiochemical properties including mineral and microbial signatures. Depending upon the need and requirement of population large consumption, on day-to-day basis makes drinking water a probable base of Gut microbial diversity.

Limited research is available on influence and impact of drinking water on Gut microbial diversity and is yet to be explored in terms of its physiological, pathological and therapeutic character on human system. However, very few researchers have studied the alteration in Gut microbial variation and alteration due to consumption of different types of water that lead to fluctuation and alteration of Gut microbiota composition. One of the researchers explored that tap water consumption increased the Gut microbiome bacterial population associated with antibiotic resistance in mice as compared to sterilized water consumption.

Present protocol is an attempt to explore the consequence of Alkaline water consumption on the Gut microbiota of doxorubicin induced toxicity/remodelling in rats. Furthermore, levels of inflammatory mediators including CRP, Interleukin-10 (IL-10), Fibrinogen, Angiotensin II (Ang II), Renin, Interleukin-6 (IL-6), troponin, TNFα, and Creatine Kinase Myocardial Band (CKMB) in plasma are also estimated along with alteration in histopathological findings on heart and kidneys.

In the view of the forgoing discussion, it is clearly portrayed that there is a need to have a system and method for treating cardiac toxicity via gut microbiota.

### SUMMARY OF THE INVENTION

The present disclosure seeks to provide a system and method for treating cardiac toxicity via gut microbiota to reduce plasma Zinc concentration, increased inflammation, cardiac remodeling and damaged renal function.

In an embodiment, a system for treating cardiac toxicity via gut microbiota is disclosed. The system includes a collection unit for procuring doxorubicin, diethyl ether, alkaline water and a plurality of subject animals. The system further includes a pre-processor unit for acclimatizing collected plurality of subject animals for 7 days standard laboratory condition of 12-hr light/dark cycle and maintaining at temperature of 22±2° C. with relative humidity of 50±5%.

The system further includes a classification processor unit for dividing the plurality of subject animals into at least four groups with six animals in each.

The system further includes a treating unit for feeding the divided subject animals with standard semi purified diet along with normal drinking water and standard semi purified diet along with Zam water p.o at libitum, or injecting with Dox i. p on first day of protocol fed with standard semi purified diet along with normal drinking water or Dox i.p. on first day of protocol thereby treating with Zam water p.o at libitum for 28 days.

The system further includes a monitoring processor unit for monitoring physiological and behavioral changes of plurality of subject animals for entire period of protocol. The system further includes an analysis processor unit for estimating a set of biochemical parameters and stool sample of the plurality of subject animals.

In one embodiment, the at least four groups is selected from a normal control (NC) fed with standard semi purified diet along with normal drinking water, Zam control (ZC) fed with standard semi purified diet along with Zam water p.o at libitum, Doxorubicin or Dox control (DC) animals are injected with 1 mg/kg bw Dox i.p. on the first day of protocol fed with standard semi purified diet along with normal drinking water and Dox+Zam (DZ) group injected with 1 mg/kg bw Dox i.p. on first day of protocol, and treated with Zam water p.o at libitum, for 28 days.

In one embodiment, the animals are on semi purified diet and body weight of the plurality of subject animals are recorded weekly and consumption of food and water is measured daily basis.

In one embodiment, the protocol is terminated on 29^{th} day, animals are sedated with diethyl ether for further process of organ harvesting and blood collection, wherein abdominal artery, punctured and blood is collected in centrifugal tubes approximately 3 ml/100 g body weight, allowed to clot at laboratory temperature for 30 minutes, centrifuged at 4° C. for 15 minutes at 4500 rpm.

In one embodiment, the supernatant serum is transferred to 1.5 ml tubes and kept at −80° C. for further analysis, wherein harvested heart and kidneys are weight, preserved in 10% phosphate buffered formaldehyde for histopathological analysis.

In one embodiment, the stool plurality of subject animals are used to isolate the DNA and briefly 250-300 mg of stool samples are collected from lower intestine, in 2 ml tube weighted and thawed on ice, wherein 1,5 ml ASL buffer is added and vortexed for 3 minutes followed by incubation at 80° C. for 15 minutes, wherein the incubated tubes are tapped every four minute and centrifuge at 14000 rpm for 15 minutes at room temperature.

In one embodiment, the EX tablet is added to 1.5-2.0 ml of supernatant, followed by centrifugation for 5 minutes at 15000 rpm, wherein the procedure continued with the addition of 15 μl proteinase K and AL buffer thereby incubated for 10 minutes at 70° C.

In one embodiment, the approximately 300 μl of absolute alcohol or ethanol is added and centrifuged for 1 minute at 12000 rpm with addition of 500 μL of AW1 buffer to column above and centrifuge at 14000 rpm for 1 minute, wherein the filtrate in collection tube is discarded and the DNA is eluted and is evaluated on 1% agarose gel thereby the isolated sample of DNA is forwarded for 16S metagenomic analysis, wherein the serum biochemical levels of Zinc, Fibrinogen, Renin, IL-10, 11-6, CRP, Ang II, and CKMB are analyzed by standard auto analyzer kits-Zinc quantification kit, Rat Fibrinogen ELISA kit, Rat Renin ELISA kit, IL-10 ELISA kit, I1-6 ELISA kit, CRP ELISA kit, Ang II ELISA kit, and CKMB ELISA kit from Abcam Cambridge, United Kingdom.

In one embodiment, the harvested organs including heart and kidneys are fixed in formalin (10%), dehydrated with alcohol for an hour in an ascending order from 70%, 80%, 90%, and 100%, wherein dehydrated tissues are subjected to treatment with xylene, fixed with molten paraffin, and freeze to solidify for an hour, wherein sections of 5 μm via automated microtome are sliced, mounted on slides, dried overnight at 37° C. and xylene is removed and deparaffined for further stained with Hematoxylin and Eosin, finally permanent slides are prepared by DPX before fixing the coverslips.

In another embodiment, a method for treating cardiac toxicity via gut microbiota is disclosed. The method includes procuring doxorubicin, diethyl ether, alkaline water and a plurality of subject animals using a collection unit. The method further includes acclimatizing collected plurality of subject animals for 7 days standard laboratory condition of 12-hr light/dark cycle and maintaining at temperature of 22±2° C. with relative humidity of 50±5% by employing a pre-processor unit. The method further includes dividing the plurality of subject animals into at least four groups with six animals in each by deploying a classification processor unit. The method further includes feeding the divided subject animals with standard semi purified diet along with normal drinking water and standard semi purified diet along with Zam water p.o at libitum, or injecting with Dox i. p on first day of protocol fed with standard semi purified diet along with normal drinking water or Dox i.p. on first day of protocol thereby treating with Zam water p.o at libitum for 28 days by a treating unit. The method further includes monitoring physiological and behavioral changes of plurality of subject animals for entire period of protocol using a monitoring processor unit. The method further includes estimating a set of biochemical parameters and stool sample of the plurality of subject animals through an analysis processor unit.

An object of the present disclosure is to reduce plasma Zinc concentration, increased inflammation, cardiac remodeling and damaged renal function.

Another object of the present disclosure is to provide protection against cardiac and renal injury via richness and diversity of Gut microbiome, healthier and diversified composition is function of Zinc concentration at given pH.

Yet another object of the present invention is to deliver an expeditious and cost-effective method for treating cardiac toxicity via gut microbiota.

To further clarify advantages and features of the present disclosure, a more particular description of the invention will be rendered by reference to specific embodiments thereof, which is illustrated in the appended drawings. It is appreciated that these drawings depict only typical embodiments of the invention and are therefore not to be considered limiting of its scope. The invention will be described and explained with additional specificity and detail with the accompanying drawings.

### BRIEF DESCRIPTION OF FIGURES

These and other features, aspects, and advantages of the present disclosure will become better understood when the following detailed description is read with reference to the accompanying drawings in which like characters represent like parts throughout the drawings, wherein:

FIG. 1 illustrates a block diagram of a system for treating cardiac toxicity via gut microbiota in accordance with an embodiment of the present disclosure;

FIG. 2 illustrates a flow chart of a method for treating cardiac toxicity via gut microbiota in accordance with an embodiment of the present disclosure;

FIGS. 3A-3D illustrate taxonomy abundance and distribution in normal control (NC) rats, alkaline water control (ZC) rats, dox control (DC) rats, dox group treated with alkaline water (DZ) in accordance with an embodiment of the present disclosure;

FIGS. 4A-4D illustrate heart histopathology image of random images in normal control (NC) group, alkaline water control (ZC) group, the doxorubicin-induced cardiac remodelling control (DC) group, the doxorubicin-induced cardiac remodelling control+Alkaline water (DZ) group in accordance with an embodiment of the present disclosure;

FIGS. 5A-5D illustrate kidney histopathology of random images in normal control (NC) group, alkaline water control (ZC) group, the doxorubicin-induced cardiac remodelling control (DC) group, the doxorubicin-induced cardiac remodelling control+alkaline water (DZ) group in accordance with an embodiment of the present disclosure;

FIG. 6 illustrates Table 1 depicts comparative analysis of alkaline water with/without disease expressed for Zinc, CRP, IL-6, IL-10, Fibrinogen, Ang II, renin and CKMB in accordance with an embodiment of the present disclosure;

FIG. 7 illustrates Table 2 depicts expression of % abundance of microbial flora in different groups in accordance with an embodiment of the present disclosure;

FIG. 8 illustrates Table 3A depicts Variations of the characteristics of heart histopathology of random images in accordance with an embodiment of the present disclosure; and

FIG. 9 illustrates Table 3B depicts variations of the characteristics of kidney histopathology of random images in normal control (NC) group, Zinc control (ZC), Dox, Control (DC), and Dox with Alkaline water (DZ) in accordance with an embodiment of the present disclosure.

Further, skilled artisans will appreciate that elements in the drawings are illustrated for simplicity and may not have necessarily been drawn to scale. For example, the flow charts illustrate the method in terms of the most prominent steps involved to help to improve understanding of aspects of the present disclosure. Furthermore, in terms of the construction of the device, one or more components of the device may have been represented in the drawings by conventional symbols, and the drawings may show only those specific details that are pertinent to understanding the embodiments of the present disclosure so as not to obscure the drawings with details that will be readily apparent to those of ordinary skill in the art having benefit of the description herein.

### DETAILED DESCRIPTION

For the purpose of promoting an understanding of the principles of the invention, reference will now be made to the embodiment illustrated in the drawings and specific language will be used to describe the same. It will nevertheless be understood that no limitation of the scope of the invention is thereby intended, such alterations and further modifications in the illustrated system, and such further applications of the principles of the invention as illustrated therein being contemplated as would normally occur to one skilled in the art to which the invention relates.

It will be understood by those skilled in the art that the foregoing general description and the following detailed description are exemplary and explanatory of the invention and are not intended to be restrictive thereof.

Reference throughout this specification to “an aspect”, “another aspect” or similar language means that a particular feature, structure, or characteristic described in connection with the embodiment is included in at least one embodiment of the present disclosure. Thus, appearances of the phrase “in an embodiment”, “in another embodiment” and similar language throughout this specification may, but do not necessarily, all refer to the same embodiment.

The terms “comprises”, “comprising”, or any other variations thereof, are intended to cover a non-exclusive inclusion, such that a process or method that comprises a list of steps does not include only those steps but may include other steps not expressly listed or inherent to such process or method. Similarly, one or more devices or sub-systems or elements or structures or components proceeded by “comprises . . . a” does not, without more constraints, preclude the existence of other devices or other sub-systems or other elements or other structures or other components or additional devices or additional sub-systems or additional elements or additional structures or additional components.

Unless otherwise defined, all technical and scientific terms used herein have the same meaning as commonly understood by one of ordinary skill in the art to which this invention belongs. The system, methods, and examples provided herein are illustrative only and not intended to be limiting.

Embodiments of the present disclosure will be described below in detail with reference to the accompanying drawings.

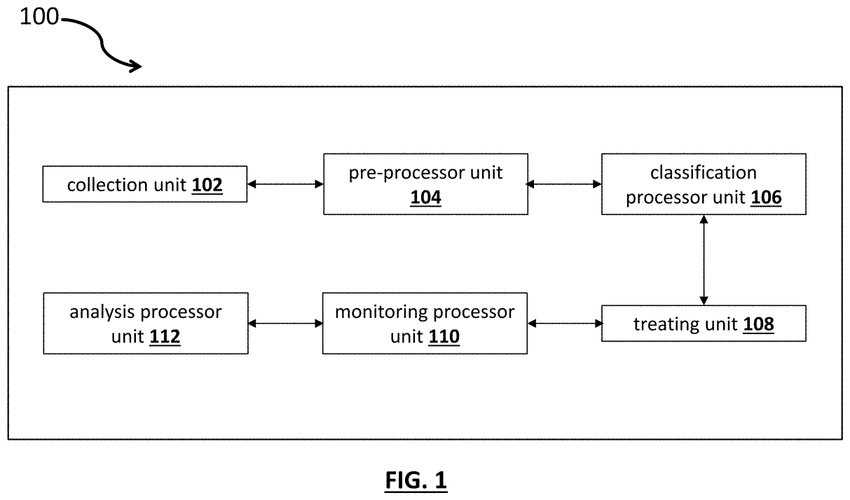

Referring to FIG. 1, a block diagram of a system for treating cardiac toxicity via gut microbiota is illustrated in accordance with an embodiment of the present disclosure. The system 100 includes a collection unit 102 for procuring doxorubicin, diethyl ether, alkaline water and a plurality of subject animals.

In an embodiment, a pre-processor unit 104 is coupled to the collection unit 102 for acclimatizing collected plurality of subject animals for 7 days standard laboratory condition of 12-hr light/dark cycle and maintaining at temperature of 22±2° C. with relative humidity of 50±5%.

In an embodiment, a classification processor unit 106 is connected to the pre-processor unit 104 for dividing the plurality of subject animals into at least four groups with six animals in each.

In an embodiment, a treating unit 108 is coupled to the classification processor unit 106 for feeding the divided subject animals with standard semi purified diet along with normal drinking water and standard semi purified diet along with Zam water p.o at libitum, or injecting with Dox i. p on first day of protocol fed with standard semi purified diet along with normal drinking water or Dox i.p. on first day of protocol thereby treating with Zam water p.o at libitum for 28 days.

In an embodiment, a monitoring processor unit 110 is connected to the treating unit 108 for monitoring physiological and behavioral changes of plurality of subject animals for entire period of protocol.

In an embodiment, an analysis processor unit 112 is coupled to the monitoring processor unit 110 for estimating a set of biochemical parameters and stool sample of the plurality of subject animals.

In one embodiment, the at least four groups is selected from a normal control (NC) fed with standard semi purified diet along with normal drinking water, Zam control (ZC) fed with standard semi purified diet along with Zam water p.o at libitum, Doxorubicin or Dox control (DC) animals are injected with 1 mg/kg bw Dox i.p. on the first day of protocol fed with standard semi purified diet along with normal drinking water and Dox+Zam (DZ) group injected with 1 mg/kg bw Dox i.p. on first day of protocol, and treated with Zam water p.o at libitum, for 28 days.

In one embodiment, the animals are on semi purified diet and body weight of the plurality of subject animals are recorded weekly and consumption of food and water is measured daily basis.

In one embodiment, the protocol is terminated on 29^{th} day, animals are sedated with diethyl ether for further process of organ harvesting and blood collection, wherein abdominal artery, punctured and blood is collected in centrifugal tubes approximately 3 ml/100 g body weight, allowed to clot at laboratory temperature for 30 minutes, centrifuged at 4° C. for 15 minutes at 4500 rpm.

In one embodiment, the supernatant serum is transferred to 1.5 ml tubes and kept at −80° C. for further analysis, wherein harvested heart and kidneys are weight, preserved in 10% phosphate buffered formaldehyde for histopathological analysis.

In one embodiment, the stool plurality of subject animals are used to isolate the DNA and briefly 250-300 mg of stool samples are collected from lower intestine, in 2 ml tube weighted and thawed on ice, wherein 1,5 ml ASL buffer is added and vortexed for 3 minutes followed by incubation at 80° C. for 15 minutes, wherein the incubated tubes are tapped every four minute and centrifuge at 14000 rpm for 15 minutes at room temperature.

In one embodiment, the EX tablet is added to 1.5-2.0 ml of supernatant, followed by centrifugation for 5 minutes at 15000 rpm, wherein the procedure continued with the addition of 15 μl proteinase K and AL buffer thereby incubated for 10 minutes at 70° C.

In one embodiment, the approximately 300 μl of absolute alcohol or ethanol is added and centrifuged for 1 minute at 12000 rpm with addition of 500 μL of AW1 buffer to column above and centrifuge at 14000 rpm for 1 minute, wherein the filtrate in collection tube is discarded and the DNA is eluted and is evaluated on 1% agarose gel thereby the isolated sample of DNA is forwarded for 16S metagenomic analysis, wherein the serum biochemical levels of Zinc, Fibrinogen, Renin, IL-10, 11-6, CRP, Ang II, and CKMB are analyzed by standard auto analyzer kits-Zinc quantification kit, Rat Fibrinogen ELISA kit, Rat Renin ELISA kit, IL-10 ELISA kit, I1-6 ELISA kit, CRP ELISA kit, Ang II ELISA kit, and CKMB ELISA kit from Abcam Cambridge, United Kingdom.

In one embodiment, the harvested organs including heart and kidneys are fixed in formalin (10%), dehydrated with alcohol for an hour in an ascending order from 70%, 80%, 90%, and 100%, wherein dehydrated tissues are subjected to treatment with xylene, fixed with molten paraffin, and freeze to solidify for an hour, wherein sections of 5 μm via automated microtome are sliced, mounted on slides, dried overnight at 37° C. and xylene is removed and deparaffined for further stained with Hematoxylin and Eosin, finally permanent slides are prepared by DPX before fixing the coverslips.

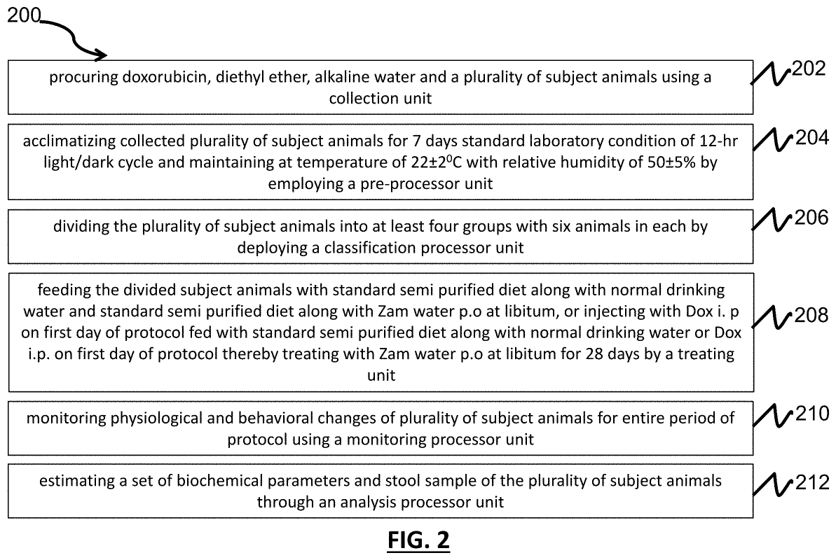

FIG. 2 illustrates a flow chart of a method for treating cardiac toxicity via gut microbiota in accordance with an embodiment of the present disclosure. At step 202, the method 200 includes procuring doxorubicin, diethyl ether, alkaline water and a plurality of subject animals using a collection unit 102.

At step 204, the method 200 includes acclimatizing collected plurality of subject animals for 7 days standard laboratory condition of 12-hr light/dark cycle and maintaining at temperature of 22±2° C. with relative humidity of 50±5% by employing a pre-processor unit 104.

At step 206, the method 200 includes dividing the plurality of subject animals into at least four groups with six animals in each by deploying a classification processor unit 106.

At step 208, the method 200 includes feeding the divided subject animals with standard semi purified diet along with normal drinking water and standard semi purified diet along with Zam water p.o at libitum, or injecting with Dox i. p on first day of protocol fed with standard semi purified diet along with normal drinking water or Dox i.p. on first day of protocol thereby treating with Zam water p.o at libitum for 28 days by a treating unit 108.

At step 210, the method 200 includes monitoring physiological and behavioral changes of plurality of subject animals for entire period of protocol using a monitoring processor unit 110.

At step 212, the method 200 includes estimating a set of biochemical parameters and stool sample of the plurality of subject animals through an analysis processor unit 112.

Drugs and Reagents Doxorubicin and Diethyl ether are procured from Sigma Aldrich, United States of America, Alkaline water Ready to consume procured and analysed.

Analysing Kits

Zinc Quantification, rat high sensitivity C-Reactive Protein, Fibrinogen invitro, IL-6, IL-10, Renin, Ang II, and CKMB Enzyme linked Immunosorbent Assay (ELISA) rat kits are procured from Abcam, Cambridge, United Kingdom.

Animal Groups

Sufficient experimental animals Wistar albino rats approximately average weight of 170±20 g are obtained. The protocol is approved by Institutional Animal Ethical Committee, all animals are acclimatized for 7 days standard laboratory condition of 12-hr light/dark cycle, maintained at temperature of 22±2° C. with relative humidity of 50±5% in department of Biochemistry, Faculty of Science.

All the animals are randomly divided into 4 groups with six animals in each: 1) Normal control (NC), fed with standard semi purified diet along with normal drinking water, 2) Zam control (ZC) fed with standard semi purified diet along with Zam water p.o at libitum, 3) Doxorubicin or Dox control (DC), animals are injected with Dox (1 mg/kg bw) i.p. on first day of protocol fed with standard semi purified diet along with normal drinking water and 4) Dox+Zam (DZ) group injected with Dox (1 mg/kg bw) i.p. on first day of protocol, and treated with Zam water p.o at libitum, for 28 days. All the animals are on semi purified diet throughout the study period. Body weight of rats are recorded weekly and consumption of food and water is measured daily basis.

Sample Collection

Physiological and behavioural changes are monitored in experimental rats for entire period of protocol. The protocol is terminated on 29^{th} day, animals are sedated with diethyl ether for further process of organ harvesting and blood collection. Abdominal artery, punctured and blood is collected in centrifugal tubes approximately 3 ml/100 g body weight, allowed to clot at laboratory temperature for 30 minutes, centrifuged at 4° C. for 15 minutes at 4500 rpm. The supernatant serum, transferred to 1.5 ml tubes and kept at −80° C. for further analysis. Harvested heart and kidneys are weight, preserved in 10% phosphate buffered formaldehyde for histopathological analysis.

Gut Microbiota Analysis

Stool samples of rats are used to isolate the DNA by QAIGEN kit as per manufacturer's instructions. Briefly 250-300 mg of stool samples collected from lower intestine, in 2 ml tube weighted and thawed on ice. ASL buffer (1.5 ml) is added, vortexed for 3 minutes followed by incubation at 80° C. for 15 minutes. The incubated tubes are tapped every four minute and centrifuge at 14000 rpm for 15 minutes at room temperature. EX tablet is added to 1.5-2.0 ml of supernatant, followed by centrifugation for 5 minutes at 15000 rpm. The procedure continued with the addition of proteinase K (15 μl) and AL buffer, incubated for 10 minutes at 70° C. Approximately 300 μl of absolute alcohol or ethanol is added, centrifuged for 1 minute at 12000 rpm with addition of 500 μl of AW1 buffer to column above and centrifuge at 14000 rpm for 1 minute. The filtrate in collection tube is discarded, finally DNA is eluted and is evaluated on 1% agarose gel. The isolated sample of DNA is forwarded for 16S metagenomic analysis.

Estimation of Biochemical Parameters

The serum biochemical levels of Zinc, Fibrinogen, Renin, IL-10,11-6, CRP, Ang II, and CKMB are analyzed by standard auto analyzer kits-Zinc quantification kit, Rat Fibrinogen ELISA kit, Rat Renin ELISA kit, IL-10 ELISA kit, I1-6 ELISA kit, CRP ELISA kit, Ang II ELISA kit, and CKMB ELISA kit from Abcam Cambridge, United Kingdom.

Water Analysis

The samples of ready to consume Zam water and bottled drinking water analyzed. Jeddah for Alkalinity and presence of other inorganic by Inductive Couple Plasma Mass Spectroscopy (ICPMS) technology. The composition of Alkaline water is as follows: (Zinc: 0-15-0.19 μg/l, pH: 7.4, Calcium: 38.30 mg/l, Bicarbonate ions Approx. 195-199.0 mg/l Total Hardness: 129.95 mg/l, TDS: 429.17, Conductance: 663.00 μs/cm,

Histopathological Analysis

Briefly, harvested organs including heart and kidneys are fixed in formalin (10%), dehydrated with alcohol for an hour in an ascending order from 70%, 80%, 90%, and 100% respectively. Dehydrated tissues are subjected to treatment with xylene, fixed with molten paraffin, freeze to solidify for an hour. Sections of 5 μm via automated microtome are sliced, mounted on slides, dried overnight at 37° C. Xylene is removed and deparaffined for further stained with Hematoxylin and Eosin, finally permanent slides are prepared by DPX before fixing the coverslips. Microscopical images are developed and observed under various magnifications.

Physical Assessment

The animals are observed for the changes in behavioral pattern if any. Body weight is measured every week during the study period. No behavioral abnormalities are noted in the animals in any of the 4 groups in given circumstances and environment with no significant alteration in body weight.

Water Analysis

The samples of Alkaline water and bottled water are analyzed by ICPMS technology for the presence of Zinc. Bottles water is significantly high in Zinc concentration compared to Zam. Bicarbonate ions (HCO₃⁻) essential to maintain the alkaline. Alkaline water (pH 7.4) as compared to bottled water (pH 6.3). Other composition of Alkaline water is Zinc: 0-15-0.19 μg/l, Calcium: 38.30 mg/l, Bicarbonate ions Approx. 195-199.0 mg/l Total Hardness: 129.95 mg/l, TDS: 429.17, Conductance: 663.00 μs/cm,

Blood Analysis

Biochemical levels of Zinc, Fibrinogen, Renin, IL-10, 11-6, CRP, Ang II, and CKMB of various experimental animals are illustrated in Table 1. There is sharp fall in significant level of Zinc (0.55±0.12 mcg/ml) in Dox group compared (0.89±0.03 mcg/ml) to NC group animals. There are sharp restoration of Zinc ions (0.72±0.07 mcg/ml) in therapeutic group on treatment with Zam water compared to Dox induced disease group. Plasma CRP level an indicator of internal inflammation is much high in DC group (13.40±2.66 mg/dL), compared to NC group (7.60±0.26 mg/dL). Therapeutic group DZ indicated a significant (p<0.05) fall in CRP level compared to DC group. IL-10 a potent anti-inflammatory marker is decreased at a significant level of 3.73±0.50 pg/mL (p<0.05) in DC at a single dose of 1 mg/kg body weight compared to NC group 8.53±0.45 pg/mL, while there is a notable change or increase 7.22±0.84 pg/mL of IL-10 is observed in therapeutic group DZ (Table 1). IL-6 pro inflammatory marker is low in 3.36±1.32 in DC group compared to NC animals. Alteration in IL-6 is non-significant in NC, ZC and DC, however the therapeutic group DZ exhibited significant increase in IL-6 level compared to DC group (p<0.05), a rare phenomenon. Elevated level of fibrinogen is a predictor of cardiovascular disease, significant elevation (p<0.05) is observed in Fibrinogen concentration 4.37±0.74 in DC group compared to 2.11±0.06 NC animals. Treatment with Z. significantly reduced the levels of Fibrinogen in DZ animals' indication a protective role of Zam in CVD. Ang II activates specific Ang II receptors, regulates cardiac contraction, impulse propagation and cell to cell communication. Its alteration specify the onset of CVD, the Ang II level in DC animals (16.00±2.26 nmol/mL/min) is significantly low in (p<0.01) compared to NC animals (24.67±1.76 nmol/mL/min), the treatment with Zₐₘ is able to restore the level of Ang-II (18.62±0.50 nmol/mL/min) that is significantly equal with the NC group animals. Renin a hormone secreted by juxta glamorous apparatus of Kidneys controls the blood pressure and maintain healthy levels of Sodium and potassium in body. Its alteration is a direct injury to cardiovascular system of organism. The plasma renin level (1.02±0.38 ng/ml/h) of DC animals is lower (1.32±0.47 ng/ml/h) than NC group animals. The treatment with Zₐₘ elevated (1.33±0.07 ng/ml/h) the level of renin in DZ animals. CKMB an important tool for diagnosis of cardiac injury/toxicity, the DC group animals exhibited the significantly elevated (p<0.01) level of CKMB read as 121.8±3.12 U/L, compared to NC group animals (108.7±2.03 U/L). Treatment with Zₐₘ significantly (p<0.01) reduced the elevated level of enzyme in DZ animals to (118.8±1.02 U/L).

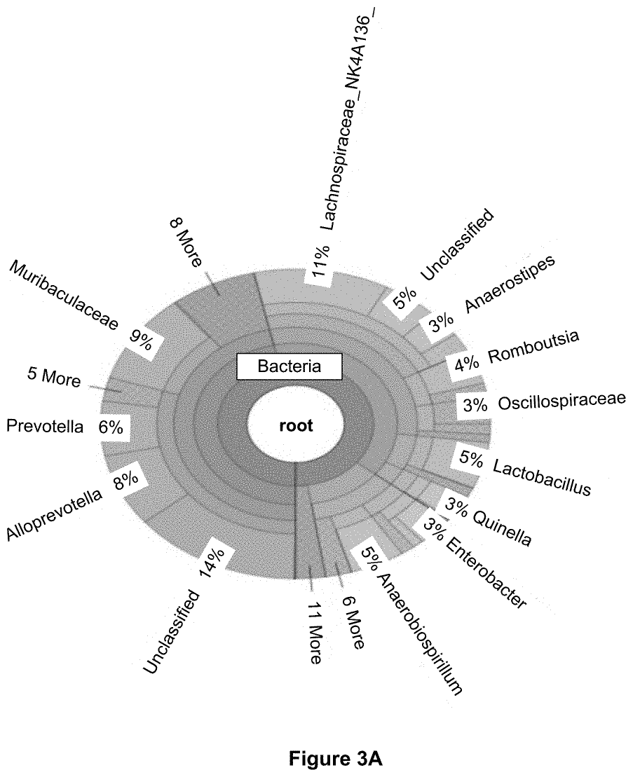

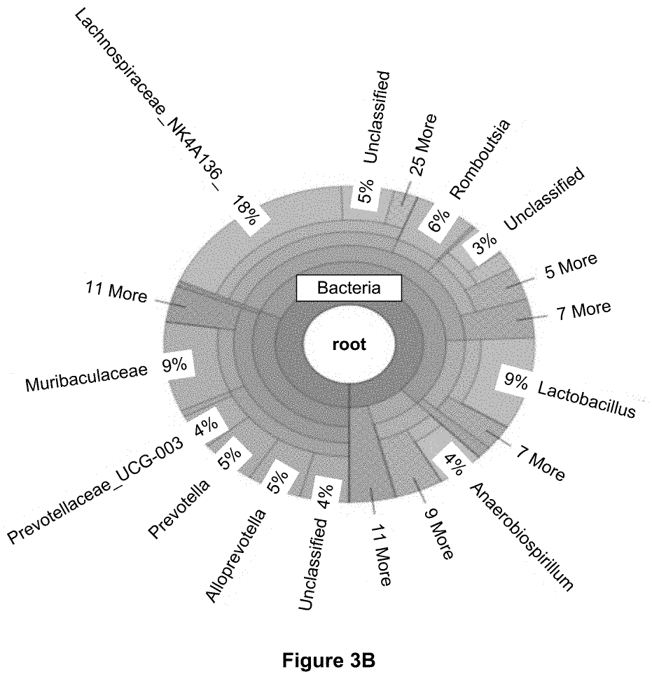

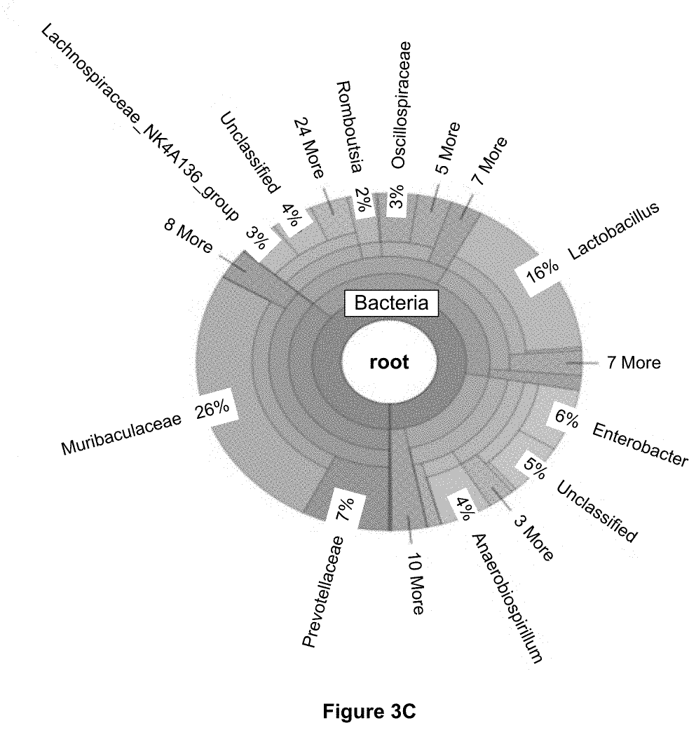

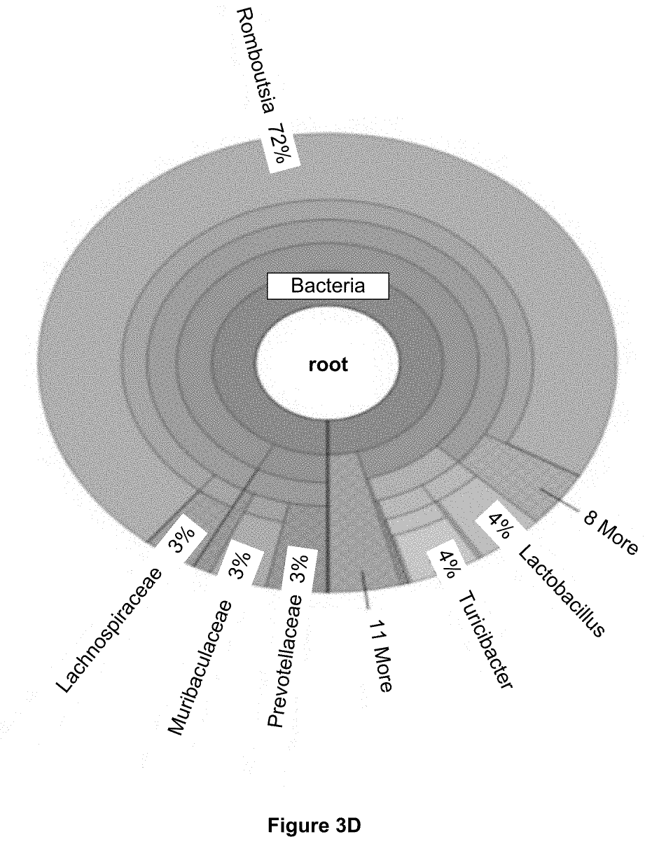

FIGS. 3A-3D illustrate taxonomy abundance and distribution in normal control (NC) rats, alkaline water control (ZC) rats, dox control (DC) rats, dox group treated with alkaline water (DZ) in accordance with an embodiment of the present disclosure. The taxonomical richness of various phyla present in the Gut microbiome of different groups are analyzed. Firmicutes and Bacteroidetes formed the majority of the community (FIG. 3A to D). It is observed that the abundance of Romboutsia, a genus of firmicutes, is just 2% in DC and 4% NC groups, however, it tremendously increased by 18-times to 72% in the group treated with Alkaline water (Table 2).

At the same time, abundance of Bacteroidetes significantly decreased in treatment group (5%) as compared to NC (45%) and DC (34%) animals. Here major shift is seen in Muribaculaceae which showed similar pattern in NC and ZC (both 9%) groups, but significantly high abundance (26%) is seen in DC group. Nevertheless, treatment with Alkaline water markedly reduced it to 3%, Similar pattern is observed in the abundance of Proteobacteria, which is markedly lower in DZ group (1%) as compared to NC (10%) and DC (17%) groups (Table 2).

FIG. 1A illustrates taxonomy abundance and distribution in Normal Control (NC) rats. FIG. 1B illustrates taxonomy abundance and distribution in Alkaline water Control (ZC) rats. FIG. 1C illustrates taxonomy abundance and distribution in Dox Control (DC) rats. FIG. 1D illustrates taxonomy abundance and distribution in Dox group treated with Alkaline water (DZ)

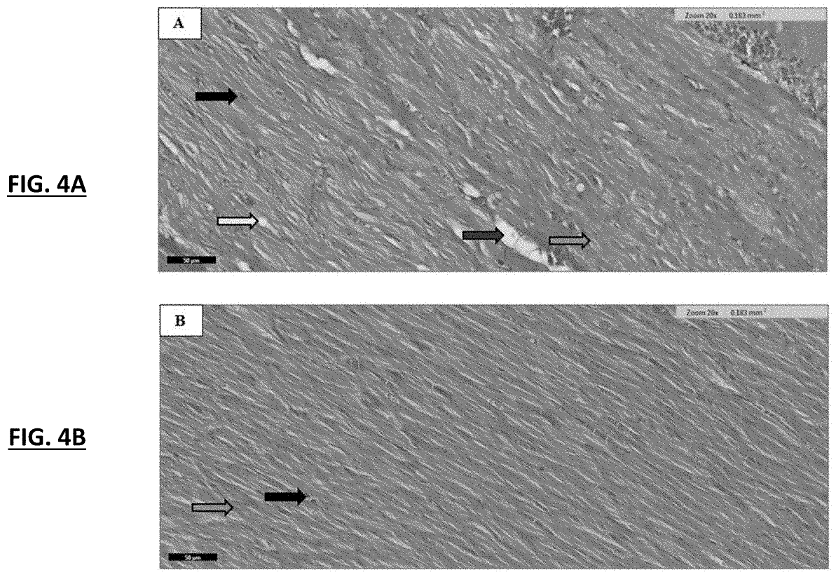

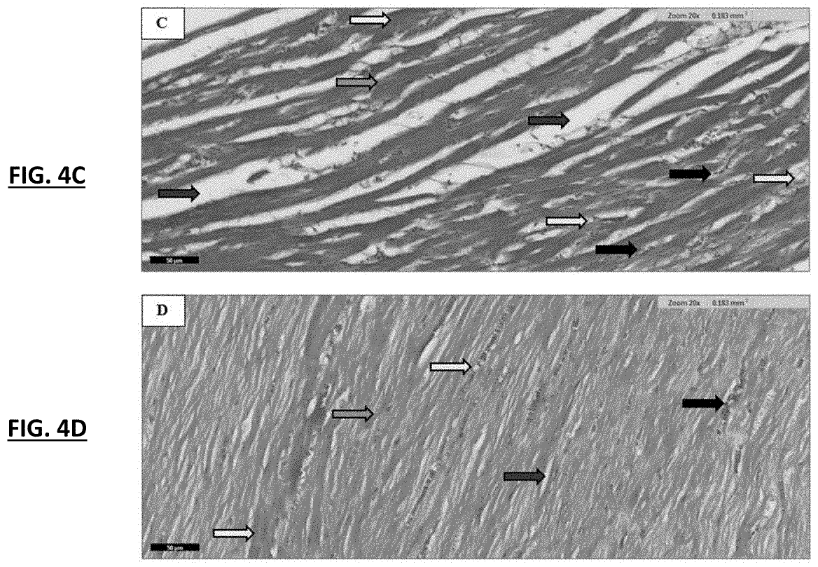

FIGS. 4A-4D illustrate heart histopathology image of random images in normal control (NC) group, alkaline water control (ZC) group, the doxorubicin-induced cardiac remodelling control (DC) group, the doxorubicin-induced cardiac remodelling control+Alkaline water (DZ) group in accordance with an embodiment of the present disclosure. All hematoxylin and eosin stained sections of kidney and heart from different experimental groups are evaluated histologically. Histopathological investigation revealed normal morphology of the tissues in Normal (NC) and Alkaline water control (ZC) animals. However, the histopathological evaluation of cardiac tissue of doxorubicin control rats showed focal areas of myocardial cell degeneration with increased intracellular spaces, high fat deposits, inflammatory cell infiltration and disrupted tissue orientation. On the other hand, marked improvement is noticed in rats treated with Alkaline water, where normal myocyte architecture and tissue orientation could be seen (FIG. 4D).

FIGS. 4A, 4B, 4C, and 4D defines the following characteristics: loose cardiac myocyte (yellow arrow), fat deposition (grey arrow), eosinophilic infiltration (black arrow), ventricular cells arrangement (blue arrow), intracellular gaps (red arrow). Hematoxylin and eosin stained; Original magnification −20X; Scale=50 μm.

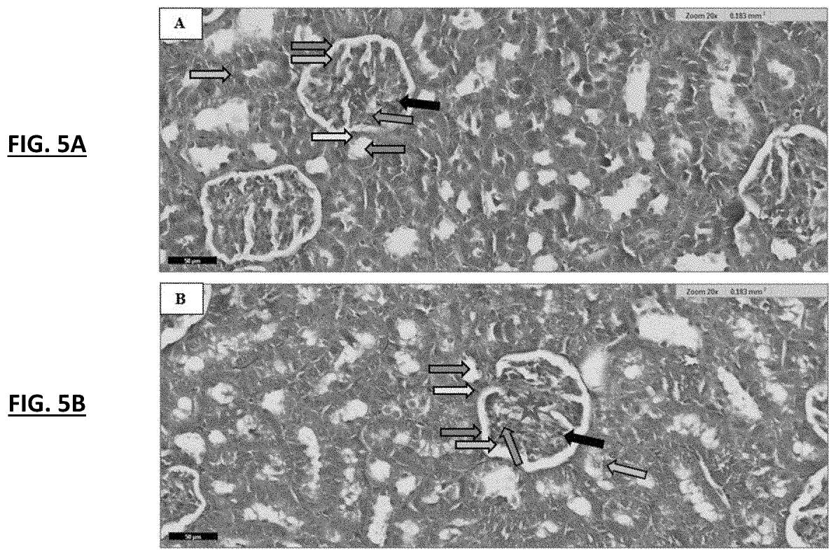

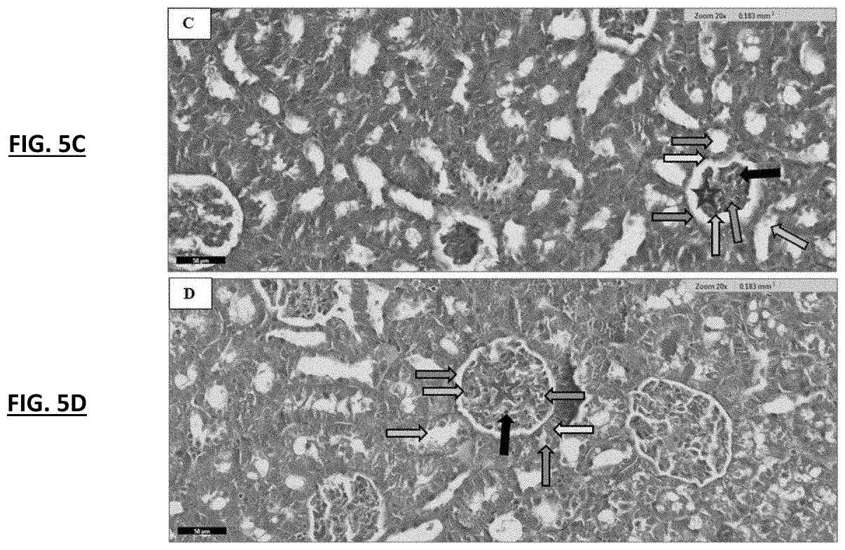

FIGS. 5A-5D illustrate kidney histopathology of random images in normal control (NC) group, alkaline water control (ZC) group, the doxorubicin-induced cardiac remodelling control (DC) group, the doxorubicin-induced cardiac remodelling control+alkaline water (DZ) group in accordance with an embodiment of the present disclosure. H&E stains of kidneys of doxorubicin treated animals showed severe pathological lesions with tubular degeneration, tubular cast and tubular necrosis with eosinophilic infiltration. Also, it reflected that doxorubicin caused swelling of tubular epithelium at the cortex, which is associated with the glomerular congestion (FIG. 5D). On the other hand, treatment with Alkaline water reduced the histopathological alterations. Detailed anatomical/architectural alteration of various groups with respect to cardiac and renal tissues are represented in Tables 3A and 3B.

The FIGS. 5A, 5B, 5C, and 5D defines the following characteristics: distribution of renal corpuscle (black star), glomerular congestion (blue star), tubular casts (grey star), proximal convoluted tubule (light green arrow), distal convoluted tubule (purple arrow), Macula densa cells (grey arrow), basal lamina (green arrow), glomerular basement membrane (orange arrow), eosinophilic infiltration (black arrow), mesangial cells (blue arrow). Hematoxylin and eosin stained; Original magnification is −20X; Scale=50 μm.

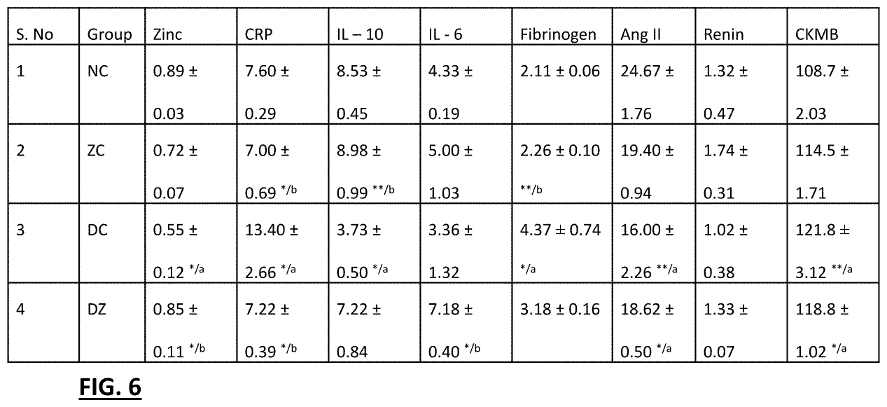

FIG. 6 illustrates Table 1 depicts comparative analysis of alkaline water with/without disease expressed for Zinc, CRP, IL-6, IL-10, Fibrinogen, Ang II, renin and CKMB in accordance with an embodiment of the present disclosure.

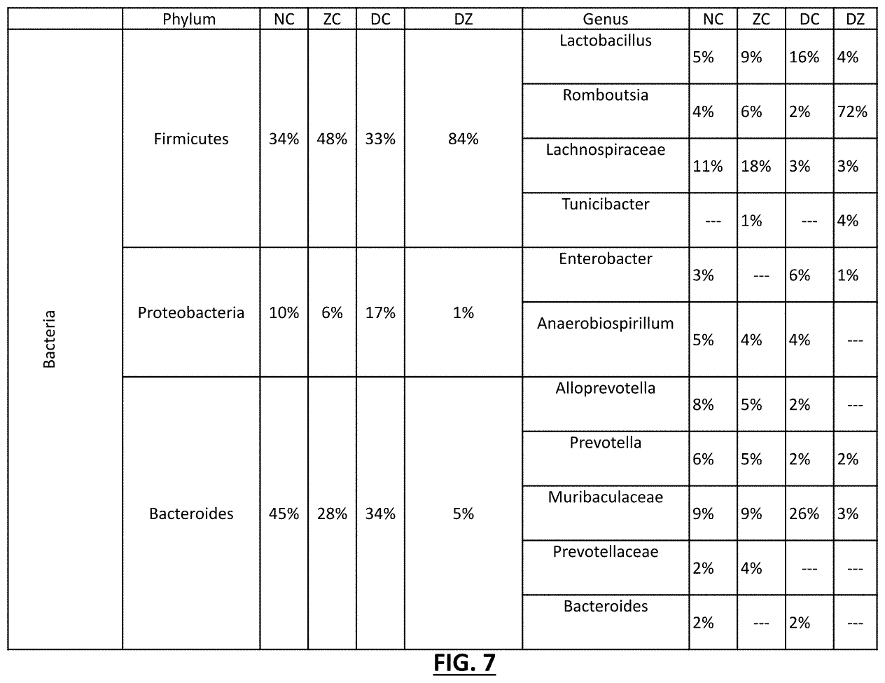

FIG. 7 illustrates Table 2 depicts expression of % abundance of microbial flora in different groups in accordance with an embodiment of the present disclosure.

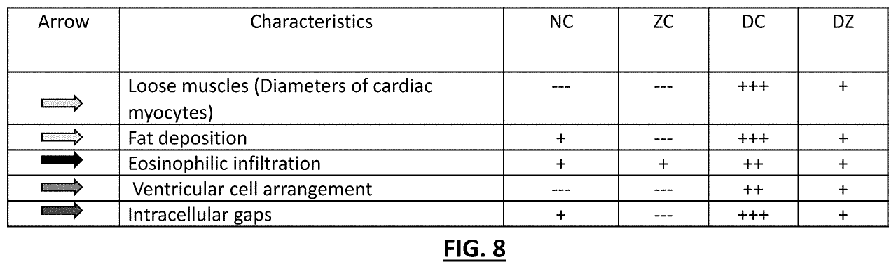

FIG. 8 illustrates Table 3A depicts Variations of the characteristics of heart histopathology of random images in accordance with an embodiment of the present disclosure.

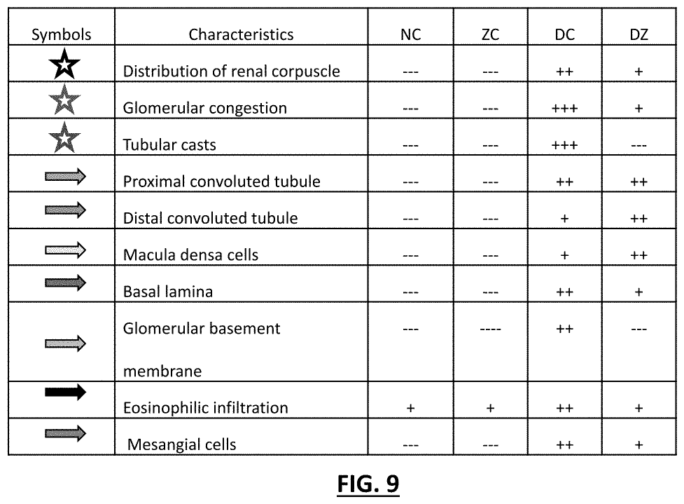

FIG. 9 illustrates Table 3B depicts variations of the characteristics of kidney histopathology of random images in normal control (NC) group, Zinc control (ZC), Dox. Control (DC), and Dox with Alkaline water (DZ) in accordance with an embodiment of the present disclosure.

In another embodiment, the doxorubicin is an anthracycline chemotherapeutic drug, with a high potential of treatment of several types of cancers. The mechanism of action demonstrates the interaction of tumor DNA gyrase topoisomerase inhibition, DNA intercalation with generation of ROS free radicals. Dose-dependent cardiac toxicity is the major adverse effect associated with the drug, which may progress to irreversible cardiomyopathy, acute pericarditis, hypertension, atherosclerosis and CHF.

The developed system 100 facilitates to decipher the role of Zₐₘ on Dox induced cardiac toxicity/remodeling in experimental animals at a dose of 1 mg/kg i.p. It is well reported in several studies that the Gut micro-genome and its metabolites are highly implicated in the progression of cardiovascular diseases. Further, it has been established that doxorubicin can modify and interferes with the normal functions and composition of Gut micro-genome. Alteration in microflora promotes and facilitates doxorubicin-induced cardiac toxicity through its influences on cellular biochemical processes and metabolism.

The origin or source of human drinking water, is a major contributing factor for variation in Gut microbiota composition. It is reported that the constituents of Zₐₘ water are stable for a long duration and not contaminated by microbial growths. At the same time, it is established various diets and water play important role in shaping human Gut microbiome. Although among food intake with balance nutrition, drinking water is consumed in highest amount by most of world population, however, very small information is available bout its role and impact on microbiome.

Zinc deficiency is often a predisposing factor for cardiovascular disorders and mortality. It is an essential cofactor, vital for normal physiological functions of heart, energy metabolism, signaling pathways and has antioxidant properties. Dietary supplementation of organic Zinc has improved intestinal health and functions of animals.

Alkaline water has been characterized for possessing various minerals and trace elements. It is reported to have 34 different elements including Zinc, which is an essential constituent of the antioxidant enzymes-superoxide dismutase, glutathione peroxidase and catalase. Previous studies have described the therapeutic potential of Alkaline water in various ailments including diabetic nephropathy, blood methemoglobin and bone density.

In another embodiment, the doxorubicin administration markedly attenuated serum Zinc level in disease animals compared to NC and ZC animals (Table 1). Treatment initiated with Zₐₘ is able to restored the level of this element to a significant level in therapeutic group animals. There exists a strong relationship between Zinc metabolism and Gut flora, making it a significant factor for Gut bacterial biodiversity. It has been observed that a significant shift in the composition of the major phyla constituting the microbiota. Firmicutes population markedly raised from 33% in DC group to 84% on administration/treatment with Alkaline water. Simultaneously, Protobacteria decreased from 17% in DC to 1%, and Bacteroides from 34% to 5% in treatment (DZ) group (Table 2), thus making Zinc a predominating factor in treatment of CVD via alteration in Gut microbiota in Alkaline water.

In another embodiment, it has been observed that the variations in composition of Gut microbiota of the mice fed with neural or acidic drinking water, change in water has a profound impact on Gut microbial taxonomy. It is already established that Zinc in alkaline pH have cardio protective characteristics. The system strongly advocate, presence of Zinc in alkaline water of Alkaline water retards or alters the translocation of metabolites secreted by Gut microbiota in systemic circulation of DZ animals and thus maintained Gut richness. Furthermore, the richness of Gut microbes might have maintained the Gut wall integrity, that is disrupted in doxorubicin control (DC) group, leaking the metabolites in systemic circulation that disturbs the normal physiological state of CVS in DC group.

Emerging evidences holds good the significant role of Gut microbiota in pathology of inflammation and modulating risks of several diseases like hypertension, cardiac remodeling and other chronic conditions. Mildly elevated plasma CRP level is an established biomarker for cardiovascular and other chronic inflammatory diseases. Researchers have explored a strong interaction between CRP and Gut microbiome. CRP is activated by Gut dysbiosis. It observed upstream molecules of plasma CRP in doxorubicin induced animals (Table 1). The elevated level of CRP, is an early indicator of inflammation caused by doxorubicin due to cardiac toxicity, and relatively, its association with shift in microbiota cannot be ruled-out. Gut protection by ingestion of Alkaline water also reduced significantly the CRP level in the treatment group.

The alteration in secretion pattern of inflammatory and anti-inflammatory cytokines like IL-6 and IL-10, doxorubicin induce cardiac injury is investigated. Significant shifts are observed in IL-6 and IL-10 concentrations between doxorubicin-control and normal animals (Table 1). Doxorubicin is well known to cause intestinal damage by inducing mucositis. The impaired intestinal barrier augments the entry of lipopolysaccharide (LPS), an endotoxin, to the blood circulation, and elevation in cytokines expression like IL-via toll like receptor 4 (TLR4). However, on the contrary, it observed that IL-6 level decreased in DC group animals, but increased in the group treated with Alkaline water. The gram-negative bacteria in the Gut micro genome contain LPS. The depletion of this bacteria due to disrupted microbial richness in DC group reduces LPS and consequently, IL-6 expression. Treatment with Alkaline water restored the bacterial density, and thus altered the levels of inflammatory marker.

LPS has also been involved in the activation of platelets via different mechanisms including aggregation, dense or alpha granule release and fibrinogen binding. Fibrinogen is a prognostic factor in endothelial cell injury, platelet aggregation and thrombus formation. Thrombosis with fibrinogen is of serious concern that underlies coronary artery diseases (CAD) more commonly than other coagulation factors. The system significantly demonstrates the expression of higher serum fibrinogen in DC group animals than that of NC animals indicates increased crosslinking of platelets and thrombogenesis leading to cardiac remodeling. Strong correlation and link between CVD and Gut microbiota is already established by the Mitra et al. Studies have demonstrated bacterial translocation from Gut to heart along with detection of Gut microbial DNA in atherosclerotic plagues. It can be related to our findings, where, dysbiosis might have also be involved in fibrogenesis in DC animals and is attenuated by Zₐₘ in DZ animals. The presently strongly postulated that Zₐₘ reduces the risk of CVD due to increase in richness of Gut microbiota.

Among some important biomarkers applied in clinical routine for detection of myocardial toxicity is CKMB, its elevated level in serum is direct marker of cardiac injury, including stress-based dysfunction and alteration ventricles size and volume. It is observed a significantly higher level of CKMB isoenzyme in the disease-controlled group of our study, which clearly indicated cardiac insult and inflammation. It is proposed that depletion of Gut microbiota in DC group alleviated doxorubicin-induced myocardial injury and cardiomyocyte apoptosis, as evidenced by raised CKMB level. Our findings align with the findings, where, composition and functional changes of Gut microbiota resulted in doxorubicin induced cardiac toxicity. However, protection of Gut diversity by Zinc in Alkaline reduces the movement or translocation of metabolic product of Gut microbiota in systemic circulation of DZ animals, resulted in a significant reduction of CKMB.

An immense interest among the research scientists has developed in establishing the cross talk and undeciphered function of Gut microbiota in health and disease. These findings are the tip of interaction between host and microbiota, that's not only complex but a bidirectional. The metabolic diseases like, hypertension, renal and GI diseases may. On one hand, a number of pathological conditions like hypertension, diabetes, renal and GI diseases may outline the composition and activities of microbiota. It is observed that Gut bacteria and their metabolites, like TMAO and SCFA influence the host homeostasis and at the same time, Gut bacteria and their metabolites, SCFA and TMAO, impact the host homeostasis and prompt or contribute to pathological alteration in organ function. Renin, produced by the kidneys, hydrolyzes angiotensinogen to Ang-I, which is further converted to Ang-II in presence ACE-2. Ang-II, an active component of Renin—Angiotensin System (RAS) and have proinflammatory activity that induce vasoconstriction leading to increase in blood pressure. It demonstrates, renin and Ang-II levels are unexpectedly low in DC animals compared to NC, clearly indicates the interference of doxorubicin in normal cardiac activities causing not only cardiac remodeling but also alteration in normal kidney function through Gut microbiota. RAS components are found in various tissues including GIT. Researchers are in opinion that Gut microbiota and their metabolites have potential effect on normal circulatory system, directly or indirectly modulates the Gut sympathetic activity and RAS.GI-RAS regulates normal intestinal physiology like digestion, sodium, glucose and water absorption via cotransport system along with epithelial secretion and gastric motility, with partial impact on intestinal fibrosis, inflammation, apoptosis and mucosal protection. Rather Gut microbiota facilitates Ang-II induction. Further, there are increasing evidences which prove that dysbiosis of Gut microbiota is strongly related to chronic kidney disease (CKD) through kidney-Gut axis. It is well reported that an alteration in Bacteroides to Firmicutes ratio result in decreased production of SCFAs and increased production of TMAO (CKD is associated with high level of Gut bacterial metabolites, that can impair, kidney functions and causes chronic inflammation with increase CRP. It progresses to renal tubule-interstitial fibrosis and glomerulosclerosis. Degenerative changes in juxtaglomerular cells limit their capability of producing and releasing renin. Inadequate renin in the systemic circulation in turn reduces angiotensinogen hydrolysis, and therefore, reduced Ang-II levels, as is seen in doxorubicin control group. Renal tissue destruction is further reflected by histological alterations as marked tissue degeneration, necrosis and massive inflammatory cells infiltration (FIG. 5A). Meanwhile, Alkaline water treatment significantly increased renin and Ang-II levels, thus reflecting healthy juxtaglomerular function which can be related to amelioration in microbiome richness. Further, its protective action reduced the histological alterations in kidney (FIG. 5D).

Histopathology provides vital information and exact evidence on the anatomy of tissue including possible mechanism of pathology and real-time output of concern organ in question particularly concerned with Gut microbiota. The result of developed system clearly marked the ameliorative effects Gut microbiota on cardiac and kidney tissues, as shown in FIGS. 4A-4D and FIGS. 5A-5D. The cardiac dysfunction characteristics, as loose cardiac myocyte (yellow arrow), fat deposition (grey arrow), eosinophilic infiltration (black arrow), ventricular cells arrangement (blue arrow), intracellular gaps (red arrow) in DC group animals are clearly marked are altered in therapeutic group DZ associated group with Zₐₘ treatment. Similarly, the therapeutic group of DZ via alteration in Gut microbiota is able reestablish the normal tissue structurers of kidneys the parameters including distribution of renal corpuscle (black star), glomerular congestion (blue star), tubular casts (grey star), proximal convoluted tubule (light green arrow), distal convoluted tubule (purple arrow), Macula dense cells (grey arrow), basal lamina (green arrow), glomerular basement membrane (orange arrow), eosinophilic infiltration (black arrow), mesangial cells (blue arrow) in establishment of present hypotheses. Further, it can be proposed that alteration in renin secretion is associated with Gut microbiota influence and alteration in angiotensinogen is function of Gut microbiota richness.

In another embodiment, it is concluded that doxorubicin induces Gut dysbiosis in animals, results in reduced plasma Zinc concentration, increased inflammation, cardiac remodeling and damaged renal function. The alkaline nature of Alkaline water maintains Gut integrity, reduce inflammation and free radical generation, and thereby provides protection against cardiac and renal injury via richness and diversity of Gut microbiome, healthier and diversified composition is function of Zinc concentration at given pH

Alkaline water at given concentration of parameters is cardio protective in experimental animals. Alkaline water at given concentration of parameters is Renal protective in experimental animals. Alkaline water at given concentration of parameters is cardio protective via alteration in Gut Microbiota in experimental animals. Alkaline water at given concentration of parameters is renal protective via alteration in Gut Microbiota in experimental animals. Alkaline water at given concentration of parameters alters angiotensinogen secretion from liver. Alkaline water at given concentration of parameters alters renin secretion from kidneys. Doxorubicin at given dose and condition alters RAS system. Gut dysbiosis is restored by Alkaline water to alkaline pH. Gut dysbiosis is restored by Alkaline water at given conductance and alkaline pH. Gut dysbiosis is restored by Alkaline water at given conductance and alkaline pH and TDS. Gut dysbiosis is restored by Alkaline water at given conductance and alkaline pH, TDS and Calcium. Gut dysbiosis is restored by Alkaline water at given conductance and alkaline pH, TDS, Calcium And bicarbonate ions. Alkaline water at given concentration of parameters restore the cellular architect of cardiac tissue in experimental animals. Alkaline water at given concentration of parameters restore the cellular architect of Renal tissue in experimental animals. Alkaline water at given concentration of parameters restore the cellular architect of cardiac tissue in experimental animals via Gut dysbiosis. Alkaline water at given concentration of parameters restore the cellular architect of renal tissue in experimental animals via Gut dysbiosis. Alkaline water at given concentration of parameters prevents inflammation of heart in experimental animals.

The drawings and the forgoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, orders of processes described herein may be changed and are not limited to the manner described herein. Moreover, the actions of any flow diagram need not be implemented in the order shown; nor do all of the acts necessarily need to be performed. Also, those acts that are not dependent on other acts may be performed in parallel with the other acts. The scope of embodiments is by no means limited by these specific examples. Numerous variations, whether explicitly given in the specification or not, such as differences in structure, dimension, and use of material, are possible. The scope of embodiments is at least as broad as given by the following claims.

Benefits, other advantages, and solutions to problems have been described above with regard to specific embodiments. However, the benefits, advantages, solutions to problems, and any component(s) that may cause any benefit, advantage, or solution to occur or become more pronounced are not to be construed as a critical, required, or essential feature or component of any or all the claims.

1. A system for treating cardiac toxicity via gut microbiota, the system comprises:
a collection unit for procuring doxorubicin, diethyl ether, alkaline water and a plurality of subject animals;
a pre-processor unit for acclimatizing collected plurality of subject animals for 7 days standard laboratory condition of 12-hr light/dark cycle and maintaining at temperature of 22±2° C. with relative humidity of 50±5%;
a classification processor unit for dividing the plurality of subject animals into at least four groups with six animals in each;
a treating unit for feeding the divided subject animals with standard semi purified diet along with normal drinking water and standard semi purified diet along with Zₐₘ water p.o at libitum, or injecting with Dox i. p on first day of protocol fed with standard semi purified diet along with normal drinking water or Dox i.p. on first day of protocol thereby treating with Zₐₘ water p.o at libitum for 28 days;
a monitoring processor unit for monitoring physiological and behavioral changes of plurality of subject animals for entire period of protocol; and
an analysis processor unit for estimating a set of biochemical parameters and stool sample of the plurality of subject animals.

2. The system as claimed in claim 1, wherein the at least four groups is selected from a normal control (NC) fed with standard semi purified diet along with normal drinking water, Zₐₘ control (ZC) fed with standard semi purified diet along with Zₐₘ water p.o at libitum, Doxorubicin or Dox control (DC) animals are injected with 1 mg/kg bw Dox i.p. on the first day of protocol fed with standard semi purified diet along with normal drinking water and Dox+Zₐₘ (DZ) group injected with 1 mg/kg bw Dox i.p. on first day of protocol, and treated with Zₐₘ water p.o at libitum, for 28 days.

3. The system as claimed in claim 1, wherein the animals are on semi purified diet and body weight of the plurality of subject animals are recorded weekly and consumption of food and water is measured daily basis.

4. The system as claimed in claim 1, wherein the protocol is terminated on 29^{th} day, animals are sedated with diethyl ether for further process of organ harvesting and blood collection, wherein abdominal artery, punctured and blood is collected in centrifugal tubes approximately 3 ml/100 g body weight, allowed to clot at laboratory temperature for 30 minutes, centrifuged at 4° C. for 15 minutes at 4500 rpm.

5. The system as claimed in claim 1, wherein the supernatant serum is transferred to 1.5 ml tubes and kept at −80° C. for further analysis, wherein harvested heart and kidneys are weight, preserved in 10% phosphate buffered formaldehyde for histopathological analysis.

6. The system as claimed in claim 1, wherein the stool plurality of subject animals are used to isolate the DNA and briefly 250-300 mg of stool samples are collected from lower intestine, in 2 ml tube weighted and thawed on ice, wherein 1,5 ml ASL buffer is added and vortexed for 3 minutes followed by incubation at 80° C. for 15 minutes, wherein the incubated tubes are tapped every four minute and centrifuge at 14000 rpm for 15 minutes at room temperature.

7. The system as claimed in claim 1, wherein the EX tablet is added to 1.5-2.0 ml of supernatant, followed by centrifugation for 5 minutes at 15000 rpm, wherein the procedure continued with the addition of 15 μl proteinase K and AL buffer thereby incubated for 10 minutes at 70° C.

8. The system as claimed in claim 1, wherein the approximately 300 μl of absolute alcohol or ethanol is added and centrifuged for 1 minute at 12000 rpm with addition of 500 μl of AW1 buffer to column above and centrifuge at 14000 rpm for 1 minute, wherein the filtrate in collection tube is discarded and the DNA is eluted and is evaluated on 1% agarose gel thereby the isolated sample of DNA is forwarded for 16S metagenomic analysis, wherein the serum biochemical levels of Zinc, Fibrinogen, Renin, IL-10, 11-6, CRP, Ang II, and CKMB are analyzed by standard auto analyzer kits-Zinc quantification kit, Rat Fibrinogen ELISA kit, Rat Renin ELISA kit, IL-10 ELISA kit, I1-6 ELISA kit, CRP ELISA kit, Ang II ELISA kit, and CKMB ELISA kit from Abcam Cambridge, United Kingdom.

9. The system as claimed in claim 1, wherein the harvested organs including heart and kidneys are fixed in formalin (10%), dehydrated with alcohol for an hour in an ascending order from 70%, 80%, 90%, and 100%, wherein dehydrated tissues are subjected to treatment with xylene, fixed with molten paraffin, and freeze to solidify for an hour, wherein sections of 5 μm via automated microtome are sliced, mounted on slides, dried overnight at 37° C. and xylene is removed and deparaffined for further stained with Hematoxylin and Eosin, finally permanent slides are prepared by DPX before fixing the coverslips.

10. A method for treating cardiac toxicity via gut microbiota, the method comprises:
procuring doxorubicin, diethyl ether, alkaline water and a plurality of subject animals using a collection unit;
acclimatizing collected plurality of subject animals for 7 days standard laboratory condition of 12-hr light/dark cycle and maintaining at temperature of 22±2° C. with relative humidity of 50±5% by employing a pre-processor unit;
dividing the plurality of subject animals into at least four groups with six animals in each by deploying a classification processor unit;
feeding the divided subject animals with standard semi purified diet along with normal drinking water and standard semi purified diet along with Zₐₘ water p.o at libitum, or injecting with Dox i. p on first day of protocol fed with standard semi purified diet along with normal drinking water or Dox i.p. on first day of protocol thereby treating with Zₐₘ water p.o at libitum for 28 days by a treating unit;
monitoring physiological and behavioral changes of plurality of subject animals for entire period of protocol using a monitoring processor unit; and
estimating a set of biochemical parameters and stool sample of the plurality of subject animals through an analysis processor unit.
